(19) United States
(12) Patent Application Publication
(10) Pub. No.: US 2025/0340500 A1
(43) Pub. Date: Nov. 6, 2025

(54) CONVERSION OF CARBON DIOXIDE AND WATER TO SYNTHESIS GAS

(71) Applicant: Topsoe A/S, Kgs. Lyngby (DK)

(72) Inventors: Per Juul DAHL, Vedbæk (DK); Troels Dahlgaard STUMMANN, Copenhagen (DK); Christian WIX, Nærum (DK)

(73) Assignee: Topsoe A/S, Kgs. Lyngby (DK)

(21) Appl. No.: 18/864,290

(22) PCT Filed: May 8, 2023
(86) PCT No.: PCT/EP2023/062129

§ 371 Date: Nov. 8, 2024

(30) Foreign Application Priority Data
May 11, 2022 (DK) PA202200442

(51) Int. Cl. C07C 29/151; C25B 1/04; C25B 1/23; C25B 9/70; C25B 15/08

(52) U.S. Cl. CPC C07C 29/1518; C25B 1/04; C25B 1/23; C25B 9/70; C25B 15/081; C25B 15/083

(57) ABSTRACT

The invention relates to a method for producing methanol via a synthesis gas produced by combining electrolysis of a water feedstock for producing a stream comprising hydrogen, and electrolysis of carbon dioxide rich stream for producing a stream comprising CO and CO₂ in which the synthesis gas has a molar ratio CO/CO₂ greater than 2. The invention relates also to a method for producing a synthesis gas by once-through co-electrolysis in a SOEC unit of a feed gas stream combining CO₂ and steam.

The invention relates to a method for producing a synthesis gas from a carbon dioxide-rich stream and a water feedstock via electrolysis, and where the synthesis gas is further converted to methanol, or synthetic fuels, or substitute natural gas (SNG). The synthesis gas may also be further converted to higher alcohols, i.e. C1-C5 alcohols.

Currently it is often inefficient and problematic to produce methanol from H₂ and CO₂, e.g. from a synthesis gas, this being a gas rich in H₂ and CO₂ and normally produced by steam reforming of a hydrocarbon feedstock such as natural gas. For methanol synthesis, a high CO₂ to CO ratio in the synthesis gas results in a larger methanol conversion reactor and more expensive downstream purification process.

For methanol production purposes, it is known to use electrolysis of water to produce H₂ and then mix it with CO₂ to form a synthesis gas. Hence, a known way of producing methanol is by taking a water feedstock and via electrolysis converting it into H₂, and then combining with a separate CO₂-rich stream and compressing for thereby forming a synthesis gas having a molar ratio H₂/CO₂ of about 3. This synthesis gas is then passed to a conventional methanol loop including conversion into methanol (CH₃OH) in a methanol synthesis reactor according to the reactions: 3 H₂+CO₂═CH₃OH+H₂O, CO+2 H₂═CH₃OH. The resulting raw methanol stream is then purified, i.e. enriched in methanol, via distillation, thereby producing a product stream with at least 98 wt % methanol as well as a separate water stream.

US 2007045125 A1 discloses a method for synthesizing synthesis gas from carbon dioxide obtained from atmospheric air or other available carbon dioxide source and water using a sodium-conducting electrochemical cell. Synthesis gas is also produced by the co-electrolysis of carbon dioxide and steam in solid oxide electrolytic cell. The synthesis gas produced may then be further processed and eventually converted into a liquid fuel suitable for transportation or other applications. This citation is at least silent on the use of a solid oxide electrolysis unit for conversion of CO₂ to a specific mixture of CO and CO₂.

US 20090289227 A1 discloses a method for utilizing CO₂ waste comprising recovering carbon dioxide from an industrial process that produces a waste stream comprising carbon dioxide in an amount greater than an amount of carbon dioxide present in starting materials for the industrial process. The method further includes producing hydrogen using a renewable energy resource and producing a hydrocarbon material utilizing the produced hydrogen and the recovered carbon dioxide. The carbon dioxide may be converted to CO by electrolysis and water to hydrogen by electrolysis. This citation is at least silent on the use of a solid oxide electrolysis unit for conversion of CO₂ to a specific mixture of CO and CO₂.

US 20180127668 A1 discloses a renewable fuel production system which includes a carbon dioxide capture unit for extracting carbon dioxide from atmospheric air, a carbon dioxide electrolyzer for converting carbon dioxide to carbon monoxide, a water electrolyzer for converting water to hydrogen, a synfuels generator for converting carbon monoxide produced by the carbon dioxide electrolyzer and hydrogen produced by the water electrolyzer to a fuel. The fuel produced can be synthetic gasoline and/or synthetic diesel. The carbon dioxide is converted to CO via an electrochemical conversion of CO₂, which refers to any electrochemical process in which carbon dioxide, carbonate, or bicarbonate is converted into another chemical substance in any step of the process. This citation is therefore at least silent on the use of a solid oxide electrolysis unit for conversion of CO₂, as well as converting the CO₂ to a specific mixture of CO and CO₂.

Küngas, Rainer, “Review—Electrochemical CO₂ reduction for CO production: Comparison of Low. And High-Temperature Electrolysis Technologies”; Journal of The Electrochemical Society, 2020, 167 044508, provides a review of state-of-the-art low-temperature, molten carbonate, and solid oxide electrolyzers for the production of CO.

Applicant's co-pending patent application WO PCT/EP2021/086999 discloses a method and a system for producing a synthesis gas from a carbon dioxide-rich stream and a water feedstock, where the synthesis gas is further converted to methanol by methanol synthesis. Electrolysis of water produces a feed stream comprising hydrogen and once-through electrolysis of carbon dioxide produces a feed stream comprising CO and CO₂. The feed streams are combined into a synthesis gas where the molar ratio CO/CO₂ is 0.2-0.6.

It has now been found that by using a combination of electrolysis steps for both a water feed and a CO₂ feed, it is possible to form a more reactive synthesis gas for subsequent methanol conversion and/or for production of hydrocarbon products such as synthetic fuels, resulting i.a. in a reduction of reactor size such as size of a methanol converter, less formation of water and not least a drastic reduction of the carbon foot-print. Furthermore, savings in terms of hydrogen consumption for particularly methanol conversion are achieved as well. Other associated benefits will become apparent from the below embodiments.

Accordingly, in a first aspect, the invention is a method for producing methanol comprising the steps of:
a) providing a first CO₂-rich stream and passing it through a first electrolysis unit for producing a first stream comprising CO and CO₂;
   separating from said first stream comprising CO and CO₂:
      a second stream comprising CO and CO₂, and
      a second CO₂-rich stream;
   and recycling the second CO₂-rich stream to said electrolysis unit;
b) providing a water feedstock and passing it through a second electrolysis unit for producing a stream comprising H₂,
c) combining said second stream comprising CO and CO₂ and said stream comprising H₂ into a synthesis gas;
d) converting said synthesis gas into said methanol;
   wherein said second stream comprising CO and CO₂ has a molar ratio CO/CO₂ greater than 2.

It would be understood, that the recycling of the second CO₂-rich stream to said first electrolysis unit, means that in step a) the electrolysis is not conducted in a once-through electrolysis unit.

It would be understood that the first CO₂-rich stream is a stream mainly containing CO₂, e.g. 99 vol. % or more CO₂.

It would be understood, that the first stream comprising CO and CO₂ is a mixture containing CO and CO₂, as the first CO₂-rich stream is converted in the first electrolysis unit.

As used herein, the term “passing it through” means that electrolysis process is occurring in the electrolysis unit, whereby at least part of e.g. the carbon dioxide is converted into CO with the help of electric current.

As used herein, the term “comprising” may also be interpreted as “comprising only”, i.e. “consists of”.

Hence, the invention enables converting part of the CO₂ to CO and then converting this together with the H₂ and the remaining CO₂ into methanol by methanol synthesis. Thereby, a superior synthesis feed to produce methanol is obtained compared to the prior art. The solution provided by the present invention is neutral on power consumption as the needed power for CO generation via electrolysis can be subtracted from the needed power for H₂ generation via electrolysis. Furthermore, the catalyst volume for downstream methanol synthesis, i.e. in a methanol conversion reactor, is further reduced. The superior synthesis gas will reduce both operating expenses (OPEX) and capital expenses (CAPEX).

In an embodiment, said synthesis gas has a module M=(H₂−CO₂)/(CO+CO₂) in the range 1.95-2.10, and a molar ratio CO/CO₂ greater than 2.

The synthesis gas used for methanol production is normally described in terms of said module M, since the synthesis gas is in balance for the methanol reaction when M=2. It would be understood that M=(H₂−CO₂)/(CO+CO₂) is calculated in term of molar percentages (molar concentrations). In typical synthesis gases for methanol production, such as synthesis gas produced by steam reforming, the synthesis gas will contain some excess hydrogen resulting in modules slightly above 2, for instance 2.05 or 2.10. In the present invention, suitably also, M is greater than 2, such as 2.05 or 2.10. Thereby, the size of the corresponding conversion unit, such as the size of the methanol synthesis reactor (methanol reactor) is further significantly reduced. In addition, significant savings in electrolysis power consumption is achieved.

As used herein, the term “suitably” or “suitable” is used interchangeably with the term “optionally” or “optional”, respectively, i.e. an optional embodiment.

Furthermore, while operation with a molar ratio CO/CO₂ higher than 0.6 or higher in e.g. once-through electrolysis of a CO₂-rich stream, entails a risk of carbon formation due to the higher content of CO in the gas, in the present invention the molar ratio CO/CO₂ in the exit gas at the outlet of the electrolysis unit in step a) i.e. the first stream comprising CO and CO₂, is maintained at 0.6 or lower for avoiding the risk of carbon formation, yet this exit gas is separated into the second CO₂-rich stream which is recycled to the inlet of the first electrolysis unit, and a CO rich product gas i.e. the second stream comprising CO and CO₂, with the molar ratio of CO/CO₂ above 2.

The higher the molar ratio of CO/CO₂, the better; for instance, the molar ratio CO/CO₂ in the second stream comprising CO and CO₂, and thereby also in the synthesis gas is 4 or 6 or 8 or 10 or 20 or even higher. Thereby, a superior synthesis gas is produced promoting the methanol synthesis downstream via the reaction CO+2 H₂═CH₃OH, rather than via the reaction 3 H₂+CO₂═CH₃OH+H₂O, while at the same time avoiding risks of carbon formation in the first electrolysis unit in step a), i.e. the first electrolysis unit being fed with the carbon dioxide-rich stream.

Accordingly, in an embodiment, the first stream comprising CO and CO₂ has a molar ratio CO/CO₂ of 0.6 or lower, such as in the range 0.2-0.6.

In an embodiment, the first CO₂-rich stream is produced by passing a carbon dioxide-feed stream, suitably carbon dioxide from an external source, through a CO₂-cleaning unit for removing impurities, such as Cl, sulfur, Si, As.

This ensures the protection of downstream units, here in particular the subsequent electrolysis. For instance, COS even in small amounts can cause problems. Normally, the amount of COS in industrial CO₂ is below the detection limit, but—in certain instances—COS has been measured in the range 10-20 ppb, which is enough to exert a detrimental effect on the electrolysis unit, resulting in a fast degeneration thereof.

In an embodiment, H₂O is added to the synthesis gas corresponding to a molar percentage of 1.5-3 mol % in the synthesis gas, when the CO₂ content in the synthesis gas is below 0.5 mol %. Accordingly, H₂O corresponding to a molar percentage between 1.5 and 3 is added to the synthesis gas if the CO₂ content has a molar percentage of <0.5. In other words, in this embodiment, the CO₂ content in the synthesis gas is below 0.5 mol %, and H₂O is added to synthesis gas corresponding to a molar percentage of 1.5-3 mol % in the synthesis gas.

The synthesis gas for methanol conversion comprises a mixture of CO, CO₂ and H₂, as well as H₂O. By adding H₂O so its content in the synthesis gas is 1.5-3% when the CO₂ content is below 0.5 mol %, it is now possible to better counterbalance the impact of not having sufficient CO₂ for methanol synthesis. While the molar ratio of CO to CO₂ in the synthesis gas is greater than 2, and the higher this ratio the better, it may often be desirable to keep the CO₂ content of the synthesis gas at a level not below 0.5 mol %, since methanol synthesis may still require the presence of at least some CO₂. The addition of water enables the generation of CO₂ via the water gas shift reaction: CO+H₂O═H₂+CO₂. By the present invention, it is easier to produce pure CO and not add CO₂; instead water is added.

When producing methanol, if one was to produce methanol from CO₂ and H₂, this comes at a much higher cost compared to methanol feed gas comprising H₂, CO and CO₂, because the reaction from CO₂ forms water compared to the reaction from CO; again, as a result of the reactions: CO₂+3 H₂═CH₃OH+H₂O, CO+2H₂═CH₃OH. The resulting water has a negative effect on the performance of the catalyst and the catalyst volumes increases with more than 100% if the CO₂ concentration is too high, e.g. 90%. Much more energy is also required for the purification of the methanol because all the water is removed by distillation.

The energy to conduct water and carbon dioxide electrolysis is more or less the same, if the energy to evaporate the water is included. Thus, from an energy point of view, generally it does not matter much if one conducts water or carbon dioxide electrolysis where the goal is to produce methanol from water and CO₂.

By the invention, the first electrolysis unit for producing a first stream comprising CO and CO₂ is suitably a solid oxide electrolysis cell unit, hereinafter also referred to as SOEC-CO₂ (electrolysis of CO₂ via SOEC).

In an embodiment, in conducting the CO₂ electrolysis in step a), the step of separating said first stream comprising CO and CO₂, comprises passing this stream through a CO-enrichment unit, e.g. in a pressure swing adsorption unit (PSA), for producing said second stream comprising CO and CO₂, and said second CO₂-rich stream.

From the CO-enrichment unit, e.g. PSA unit, the second stream comprising CO and CO₂ is rich in CO, thus having a molar ratio of CO/CO₂ greater than 2, and containing e.g. above 99% CO. The second CO₂-rich stream is withdrawn from the PSA at low pressure, and therefore, it is compressed and recycled to the first electrolysis unit.

The electrolysis of CO₂ to CO in step a) suitably comprises five sections in order to produce the second stream comprising CO and CO₂ with a molar ratio CO/CO₂ greater than 2, in particular high purity CO, for instance 99.9995% CO, namely: feed system, electrolysis, compression, purification (CO-enrichment) e.g. in a PSA incl. recycle compression, polishing.

The CO-enrichment unit may also be a membrane unit.

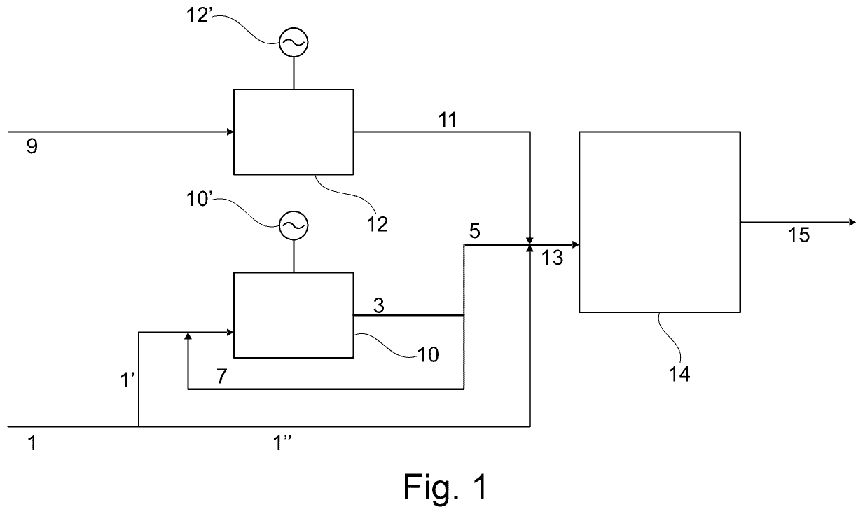

In an embodiment, the step of providing a first CO₂-rich stream and passing it through a first electrolysis unit for producing a first stream comprising CO and CO₂, and the step of providing a water feedstock and passing it through a second electrolysis unit for producing a stream comprising H₂, are conducted separately, i.e. each step is conducted with its corresponding electrolysis unit, as illustrated in appended FIG. 1.

A higher efficiency when converting the synthesis gas into methanol is achieved: when conducting co-electrolysis i.e. when the first and second electrolysis unit is the same, there will be some formation of methane as hydrogen and carbon monoxide may react. For methanol production, methane is an inert so there is an efficiency loss associated with the generation of methane.

In addition, by conducting the electrolysis of carbon dioxide and electrolysis of water separately, it is easier to optimize the e.g. SOEC stacks of the corresponding electrolysis units and the process for the two different productions.

In an embodiment, the step a) comprises by-passing a portion of said a first CO₂-rich stream prior to passing it through said first electrolysis unit, suitably a solid oxide electrolysis unit (SOEC-CO₂).

Thereby, increased flexibility in the tailoring of the molar ratio CO/CO₂ in the first stream comprising CO and CO₂ is possible, while at the same time enabling a smaller solid oxide electrolysis cell unit compared to where no by-pass is provided. For instance, the by-passed portion of the first CO₂-rich stream mainly containing CO₂ is combined with the stream exiting the first electrolysis unit, suitably after separating the second CO₂-rich stream used for recycle, together with the feed stream comprising H₂ for thereby producing said synthesis gas having thee molar ratio CO/CO₂>2 and the module M=(H₂+CO₂)/(CO−CO₂) in the range 1.95-2.10, suitably 2.05 or 2.10, as also illustrated in appended FIG. 1.

In an embodiment, the first electrolysis unit is a solid oxide electrolysis unit (herein also referred to as SOEC-CO₂ or SOEC-CO₂ unit), and the second electrolysis unit for producing the stream comprising H₂ is: an alkaline/polymer electrolyte membrane electrolysis unit i.e. alkaline and/or PEM electrolysis unit; or a solid oxide electrolysis cell unit. (SOEC unit).

The combination of using electrolysis of CO₂ via SOEC (SOEC-CO₂) and electrolysis of water via alkaline/PEM electrolysis further results in electrolysis power reduction compared to the prior art only using electrolysis of water via alkaline/PEM electrolysis with no electrolysis of CO₂.

Furthermore, when the electrolysis of H₂O to H₂ is based on liquid water (like alkaline/PEM), the heat of evaporation of the water is saved.

SOEC-CO₂ and alkaline/PEM electrolysis units are well known in the art, in particular alkaline/PEM electrolysis. For instance, applicant's WO 2013/131778 describes SOEC-CO₂.

The particular combination of SOEC-CO₂ and alkaline/PEM electrolysis is easily accessible and thereby also more inexpensive than other combinations of electrolysis units.

Particularly, in the SOEC-CO₂, CO₂ is converted to a mixture of CO and CO₂ at the fuel electrode i.e. cathode. Also, oxygen is formed at the same time at the oxygen electrode, i.e. anode, often using air as flushing gas. Thus, CO and O₂ are formed on each side of the electrolysis cell.

The present invention enables converting one mole of CO₂ to CO, thereby reducing the need for H₂ for the conversion to methanol by up to one mole, in line with the above reactions for producing methanol, which for the sake of completeness are hereby recited again: CO+2 H₂═CH₃OH; CO₂+3 H₂═CH₃OH+H₂O.

Thus, every time one mole of CO₂ is converted to one mole CO, one mole of H₂ less is needed. This conveys a significant saving in hydrogen consumption.

In a particular embodiment, the second electrolysis unit for producing the feed stream comprising H₂ is a solid oxide electrolysis cell unit. Accordingly, both the first and the second electrolysis units are solid oxide electrolysis cell units (SOEC units). Either of these electrolysis units operates suitably in the temperature range 700-800° C., which thereby enables operating with a common system for the cooling of streams thereof and thus integration of process units. Furthermore, when using SOEC both for electrolysis of CO₂ and for electrolysis of H₂O into H₂ based on steam, the energy for distillation of H₂O out of the produced CH₃OH is saved.

Operation with SOEC units at such high temperatures (700-800° C.) provides advantages over alkaline/PEM electrolysis, which operate at much lower temperature, i.e. in the range 60-160° C. Such advantages include, for instance in connection with CO₂ electrolysis, lower operational expenses due to lower cell voltage as well as lower capital expenses to higher current densities.

In an embodiment, said water feedstock comprises steam produced from other processes of the method, such as from steam generation or downstream distillation. In other words, the method of the invention may further comprise a step of producing steam from other processes of the method.

Energy efficiency of the process (method) is thereby increased since any steam generated during e.g. downstream process may be reused instead of e.g. requiring steam-export. Also, in the enrichment or purification of e.g. methanol by distillation, water is also formed which advantageously can be reused as part of the water feedstock.

It would be understood that liquid water cannot be passed through an SOEC unit, while steam cannot be passed through an alkaline/PEM unit. In other words, a SOEC unit operates with liquid water (water), while an alkaline/PEM unit operates with steam.

It would also be understood that there will be an overall saving when using water (steam) SOEC for producing H₂ if excess steam is available. Then the evaporation energy is saved in a SOEC which not will be the case if the excess steam is used for power production where the condensation heat will be lost. In particular, there will be excess steam available in the case where the end product is raw methanol, for instance where the raw methanol is produced according to Applicant's U.S. Pat. No. 4,520,216 i.e. methanol-to-gasoline route (TiGAS), where raw methanol is converted to gasoline, or any other transportation fuel such as jet fuel, or if the synthesis gas is used for substitute natural gas (SNG).

In an embodiment, said carbon dioxide feed stream or said first carbon dioxide-rich stream comprises carbon dioxide from external sources such as from biogas upgrading or fossil fuel-based synthesis gas plants.

External sources, as mentioned above, include biogas upgrade. Biogas is a renewable energy source that can be used for heating, electricity, and many other operations. Biogas can be cleaned and upgraded to natural gas standards, when it becomes bio-methane. Biogas is primarily methane (CH₄) and carbon dioxide (CO₂), typically containing 60-70% vol. methane. Up to 30% or even 40% of the biogas may be carbon dioxide. Typically, this carbon dioxide is removed from the biogas and vented to the atmosphere in order to provide a methane rich gas for further processing or to provide it to a natural gas network. The removed CO₂ is utilized for making more synthesis gas (syngas) with the method according to the present invention.

An example of a fossil fuel-based syngas plant is a natural gas-based syngas plant for gasoline production (TiGAS) i.e. a Gas-to-Liquid (GTL) process, or for methanol production where CO₂ is extracted from waste heat sections or fired heater flue gases and utilized for making more syngas with the method according to the present invention.

Other external sources include heat and power plants and waste incineration plants.

In an embodiment according to the first aspect of the invention, the electrical power required in the step of electrolysis of the carbon dioxide-rich stream or the water feedstock, is provided at least partly by renewable sources, such as wind and solar energy, or for instance also by hydropower. Thereby an even more sustainable i.e. “greener” method (process) and system (plant) approach is achievable, since no fossil fuels are used for the generation of power needed for the electrolysis. Optionally, the electricity is provided from a thermonuclear source.

In an embodiment, the step of converting the synthesis gas into methanol comprises passing the synthesis gas through a methanol synthesis reactor under the presence of a catalyst for producing a raw methanol stream, said step optionally further comprising a distillation step of the raw methanol stream for producing a water stream and a separate methanol stream having at least 98 wt % methanol.

The molar ratio of CH₃OH/H₂O in the raw methanol stream according to the present invention is 1.2 or higher, for instance 1.3 or higher, as a result of the methanol synthesis gas being more reactive than in conventional methanol synthesis or where only water electrolysis is used for producing hydrogen. In conventional methanol synthesis, from the so-called methanol loop a raw methanol product is produced having a molar ratio CH₃OH/H₂O of often about 1, which represents the production of a substantial amount of water which needs to be separated downstream. Hence, the present invention further enables that the produced raw methanol has a much lower content of water, e.g. at least 20% or at least 30% less water on a molar base, compared to conventional methanol synthesis, thereby enabling less water being carried on in the process with attendant reduction in e.g. equipment size, such as piping, as well as reducing the costs of water separation downstream, e.g. by enabling a much simpler and cost efficient distillation for the purification of methanol. Furthermore, the catalyst performance in the methanol synthesis reactor is also sensitive to water, so catalyst volumes and thereby reactor size are further reduced significantly.

Methanol technology including methanol synthesis reactors and/or methanol synthesis loops are well-known in the art. Thus, the general practice in the art is conducting the methanol conversion in a once-through methanol conversion process; or to recycle unconverted synthesis gas separated from the reaction effluent and dilute the fresh synthesis gas with the recycle gas. The latter typically results in the so-called methanol synthesis loop, herein interchangeably referred to as methanol loop, with one or more reactors connected in series or in parallel. For instance, serial synthesis of methanol is disclosed in U.S. Pat. Nos. 5,827,901 and 6,433,029, and parallel synthesis in U.S. Pat. No. 5,631,302 and EP 2874738 B1.

The method of the present invention is preferably absent of steam reforming of a hydrocarbon feed gas such as natural gas for producing the synthesis gas. Steam reforming, e.g. conventional steam methane reforming (SMR) or autothermal reforming (ATR) are large and energy intensive processes, hence operation without steam reforming for producing the synthesis gas enables significant reduction in plant size and operating costs as well as significant energy savings. In addition, compared to SMR, with electrolysis units the production capacity can easily be altered by removing or adding more electrolysis units (linear scaling of costs with size). This is normally not the case for e.g. SMR.

In a second aspect, the invention relates also to a method for producing a synthesis gas, useful for a variety of downstream applications.

Accordingly, there is provided a method for producing a synthesis gas, comprising:
i) providing a first CO₂-rich stream, providing a water feedstock and combining it with the first CO₂-rich stream to form a combined feed gas stream, and passing the combined feed gas stream through an electrolysis unit for producing said synthesis gas which comprises CO, CO₂ and H₂;
   wherein the synthesis gas has a module M=(H₂−CO₂)/(CO+CO₂) in the range 1.95-2.10, or a molar ratio H₂/CO of 1.95-2.10;
   wherein i) is conducted by once-through co-electrolysis, i.e. once-through operation in an electrolysis unit, such as a solid oxide electrolysis cell unit (SOEC unit); and
   wherein the once-through co-electrolysis is conducted in a solid oxide electrolysis cell unit and the method comprises: by-passing a portion of said first CO₂-rich stream prior to passing it through said solid oxide electrolysis cell unit.

Hence, co-electrolysis in e.g. a once-through SOEC unit is conducted by adding steam to the carbon dioxide (first CO₂-rich stream) before the SOEC unit in order to produce all or a part of the H₂ by H₂O electrolysis together with the CO₂ electrolysis. Thereby, via once-through operation, it is possible to reach the desired CO/CO₂ ratio without risk of carbon formation even where the molar ratio CO/CO₂ is higher than 0.6, due to the presence of H₂O and H₂. Furthermore, rather than providing a separate water feedstock which is introduced separately to the SOEC-unit, the water feedstock, which is steam, is combined directly with the first CO₂-rich stream, e.g. the steam is admixed thereto, thus providing a simpler solution requiring among other things less piping.

In an embodiment according to the second aspect of the invention, the synthesis gas has a molar ratio CO/CO₂ greater than 0.2, such as 0.2-0.6, or higher, such as 1 or 2 or higher, such as greater than 2. Hence, in a particular embodiment according to the second aspect of the invention, the synthesis gas has a molar ratio CO/CO₂ greater than 2.

The benefits associated with the avoidance of risk of carbon formation at high CO/CO₂ molar ratios, i.e. higher than 0.6, such as 1 or 2 or higher e.g. 10, outweigh in this second aspect of the invention the disadvantage of generating methane during the co-electrolysis, due to the reaction of hydrogen and carbon monoxide. While for downstream methanol production, methane is an inert so there is an efficiency loss associated with the generation of methane, for downstream production of for instance methane, such as substitute or synthetic natural gas (SNG), the generation of methane in co-electrolysis may in fact be advantageous.

A simpler process and plant for producing the synthesis gas is provided, compared to e.g. implementing a first electrolysis unit for generation CO from a first CO₂ and a second electrolysis unit for generating H₂ from a water feedstock (water or steam).

By providing once-through operation of e.g. the SOEC unit, there is no recycling of synthesis gas being produced. Thereby there is at least no need for a recycle compressor and thereby also no need for associated valves, pipes and control system. Attendant operating expenses such as electric power needed for the compressor as well as maintenance of the recycle compressor and the other equipment (such as valves and pipes), are thereby saved. Moreover, the need for a CO-enrichment unit such as a PSA unit may also be eliminated, thereby significantly further simplifying the process and plant for producing the synthesis gas.

As in connection with the first aspect of the invention, the term “comprising” may also be interpreted as “comprising only”, i.e. “consists of”.

As in connection with the first aspect of the invention, the term “suitably” or “suitable” is used interchangeably with the term “optionally” or “optional”, respectively, i.e. an optional embodiment.

In an embodiment according to the second aspect of the invention, said synthesis gas has a molar ratio CO/CO₂ of 0.2-0.6, and the method further comprises:
ii) subjecting the synthesis gas to a reverse water gas shift step (rWGS step).

Hence, the once-through co-electrolysis is conducted with the molar CO/CO₂ ratio in the synthesis gas being 0.6 or below, suitably 0.2-0.6, to avoid the risk of carbon formation, followed by, optionally after adding hydrogen, a rWGS step to reach the desired CO/CO₂ ratio, e.g. to reach a desired molar ratio CO/CO₂>2, via the endothermic rWGS reaction CO₂+H₂═CO+H₂O.

In a particular embodiment, the rWGS is conducted in an electrically heated WGS reactor (e-rWGS reactor). The carbon footprint of the process is thereby kept low, since apart from e.g. the SOEC unit in step i), the e-rWGS reactor in step ii) is also powered by electricity. For details on e-RWGS, details are provided in applicant's WO 2021110806.

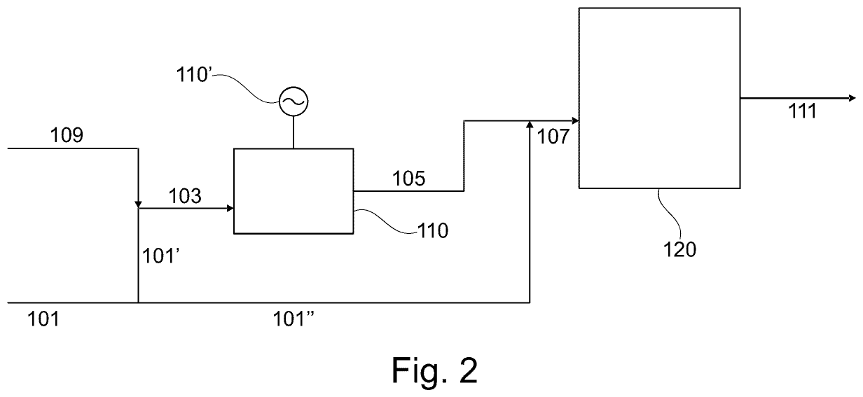

According to the second aspect of the invention, the once-through co-electrolysis is conducted in a solid oxide electrolysis cell unit and the method comprises by-passing a portion of said first CO₂-rich stream prior to passing it through said solid oxide electrolysis cell unit (SOEC-unit). The portion of the first CO₂-rich stream which is not bypassed may thus be combined with steam to generate the combined feed gas stream to the SOEC-unit, as illustrated in appended FIG. 2.

As in connection with the first aspect of the invention, this further enables increased flexibility in the tailoring of the molar ratio CO/CO₂ in the synthesis gas, while at the same time enabling a smaller solid oxide electrolysis cell unit compared to where no by-pass is provided.

In an embodiment according to the second aspect of the invention, the method further comprises:
iii) converting said synthesis gas into methanol, or synthetic fuels via Fischer Tropsch synthesis (FT synthesis), or methane e.g. substitute natural gas (SNG).

A variety of useful products are thereby obtained from the synthesis gas, all of which may be seen as renewable products or as electro fuels i.e. e-fuels. A synthesis gas with a molar ratio H₂/CO of about 2 is suitable for producing synthetic fuels (synfuels) such as jet fuel and diesel via FT-synthesis. A synthesis gas with M of about 2 and molar ratio CO/CO₂>2, such as about 10 or higher is suitable for producing methanol. The synthesis gas may also be converted to SNG via the methanation reaction whereby carbon dioxide and hydrogen react under production of methane and water: CO₂+4 H₂O═CH₄+2 H₂O.

In a particular embodiment, the method comprises converting the synthesis gas into methanol, and H₂O is added to the synthesis gas corresponding to a molar percentage of 1.5-3 mol % in the synthesis gas, when the CO₂ content in the synthesis gas is below 0.5 mol %. Accordingly, H₂O corresponding to a molar percentage between 1.5 and 3 is added to the synthesis gas if the CO₂ content has a molar percentage of <0.5. In other words, in this embodiment, the CO₂ content in the synthesis gas is below 0.5 mol %, and H₂O is added to synthesis gas corresponding to a molar percentage of 1.5-3 mol % in the synthesis gas.

As explained in connection with the first aspect of the invention, normally the synthesis gas for methanol conversion comprises a mixture of CO, CO₂ and H₂, as well as H₂O. By adding H₂O so its content in the synthesis gas is 1.5-3% when the CO₂ content is below 0.5 mol %, it is now possible to better counterbalance the impact of not having sufficient CO₂ for methanol synthesis. While the molar ratio of CO to CO₂ in the synthesis gas is greater than 2, and the higher this ratio the better, it may often be desirable to keep the CO₂ content of the synthesis gas at a level not below 0.5 mol %, since methanol synthesis still requires the presence of at least some CO₂. The addition of water enables the generation of CO₂ via the water gas shift reaction: CO+H₂0═H₂+CO₂.

As in connection with the first aspect of the invention, there is also envisaged in this second aspect of the invention that the first CO₂-rich stream is produced by passing a carbon dioxide-feed stream, suitably carbon dioxide from an external source, through a CO₂-cleaning unit for removing impurities, such as Cl, sulfur, Si, As; since this ensures the protection of downstream units, here in particular the subsequent once-through SOEC unit. For instance, COS even in small amounts can cause problems. Normally, the amount of COS in industrial CO₂ is below the detection limit, but—in certain instances—COS has been measured in the range 10-20 ppb, which is enough to exert a detrimental effect on the electrolysis unit, resulting in a fast degeneration thereof.

In a third aspect of the invention, there is also provided a method for producing higher alcohols and methane.

Accordingly, there is also provided a method for producing an alcohol, said alcohol being at least one of C1-C5 alcohols, or for producing methane, comprising the steps of:
a) providing a first CO₂-rich stream and passing it through a first electrolysis unit for producing a first stream comprising CO and CO₂;
   separating from said first stream comprising CO and CO₂:
   a second stream comprising CO and CO₂, and
   a second CO₂-rich stream;
      and recycling the second CO₂-rich stream to said first electrolysis unit;
b) providing a water feedstock and passing it through a second electrolysis unit for producing a stream comprising H₂,
c) combining said second stream comprising CO and CO₂ and said stream comprising H₂ into a synthesis gas;
d) converting said synthesis gas into said alcohol or said methane, e.g. substitute natural gas (SNG);
   wherein said second stream comprising CO and CO₂ has a molar ratio CO/CO₂ greater than 2.

The alcohol is for instance ethanol (C2 alcohol), propanol (C3 alcohol), butanol (C4), or a combination thereof.

The methane is for instance provided as substitute natural gas (SNG).

It would be understood, that any other of the embodiments and associated benefits of the first aspect of the invention related to embodiments where once-through co-electrolysis is applicable, may be combined with the second aspect of the invention, or vice-versa. Further, any of the embodiments and associated benefits of the first aspect of the invention may be combined with the third aspect of the invention, or vice-versa.

FIG. 1 shows a schematic method and system producing synthesis gas and further conversion to methanol, according to an embodiment of the first aspect of the invention.

FIG. 2 shows a schematic method and system for producing synthesis gas and further conversion to useful products, according to an embodiment of the second aspect of the invention.

With reference to FIG. 1, a carbon dioxide-feed stream such as carbon dioxide from an external source, is passed through a CO₂-cleaning unit (not shown) for removing impurities and producing a first CO₂-rich stream 1, 1′, 1″ and then through a first electrolysis unit 10 such as a SOEC-CO₂ unit i.e. CO₂-electrolysis in a SOEC unit, which is powered 10′ by a sustainable source such as wind or solar energy, thereby producing a first stream 3 comprising CO and CO₂, suitably with a molar ratio CO/CO₂ of 0.6 or below for avoiding the risk of carbon formation. This stream is separated, for instance via a PSA unit (not shown), into a second stream 5 comprising CO and CO₂ now with a molar ratio CO/CO₂>2, as well as a second CO₂-rich stream 7 which is recycled to the electrolysis unit 10. Separately, a water feedstock 9 passes through a second electrolysis unit 12, such as a PEM-electrolysis unit or SOEC unit, also powered 12′ by a sustainable source, thereby producing a stream 11 comprising H₂. Both streams 5 and 11 are combined into, compared to the prior art, a more reactive synthesis gas stream 13 having a module M=(H₂−CO₂)/(CO+CO₂) of e.g. 2.05 or 2.10, while at the same time having a molar ratio CO/CO₂>2, which is highly suitable for the downstream conversion into methanol. A portion 1″ of the first CO₂-rich stream 1 may bypass the first electrolysis unit 10, as depicted in the figure. The synthesis gas 13 enters the methanol section such as methanol loop 14 as is well-known in the art, whereby it is converted to a raw methanol stream 15 now having a molar ratio CH₃OH/H₂O of 1.3 or higher, i.e. at least 30% less water on a molar basis compared to the prior art, where the CH₃OH/H₂O ratio is normally about 1. The water in the raw methanol stream 15 is then more expediently removed in a distillation unit arranged downstream (not shown), where this stream is purified or enriched in methanol. The downstream section 14 may also be a section in which synthesis gas 13 is converted to a higher alcohol, i.e. at least one of C1-C5 alcohol, such as ethanol. The downstream section 14 may also be a section in which synthesis gas 13 is converted to methane e.g. SNG.

Now with reference to FIG. 2, a carbon dioxide-feed stream such as carbon dioxide from an external source, is passed through a CO₂-cleaning unit (not shown) for removing impurities and producing a first CO₂-rich stream 101, 101′, 101″. A water feedstock, here specifically steam 109, is added to form a combined feed gas stream which is then passed to a once-through SOEC unit 110 powered 110′ by a sustainable source such as wind or solar energy. Thereby, once-through co-electrolysis is conducted for producing a synthesis gas stream 105. By once-through co-electrolysis, the desired molar ratio of CO/CO₂ in the synthesis gas, such as >2 is obtained without risk of carbon formation due to the presence of H₂O and H₂ in the gas. A portion 101″ of the first CO₂-rich stream 101 may bypass the once-through SOEC unit 110, as depicted in the figure. The synthesis gas 105 or 107 enters a downstream section 120 such as a methanol section for producing methanol as in FIG. 1, or a Fischer Tropsch section for producing synfuels such as jet fuel or diesel, or section for converting the synthesis gas into methane, e.g. SNG.

1. A method for producing methanol comprising the steps of:
a) providing a first CO₂-rich stream and passing the first CO₂-rich stream through a first electrolysis unit for producing a first stream comprising CO and CO₂;
separating from said first stream comprising CO and CO₂:
a second stream comprising CO and CO₂; and
a second CO₂-rich stream; and
recycling the second CO₂-rich stream to said first electrolysis unit;
b) providing a water feedstock and passing the water feedstock through a second electrolysis unit for producing a stream comprising H₂;
c) combining said second stream comprising CO and CO₂ and said stream comprising H₂ into a synthesis gas; and
d) converting said synthesis gas into said methanol,
wherein said second stream comprising CO and CO₂ has a molar ratio CO/CO₂ greater than 2.

2. The method according to claim 1, wherein said synthesis gas has a module M=(H₂−CO₂)/(CO+CO₂) in the range 1.95-2.10, and a molar ratio CO/CO₂ greater than 2.

3. The method according to claim 1, wherein the first stream comprising CO and CO₂ has a molar ratio CO/CO₂ of 0.6 or lower.

4. The method according to claim 1, wherein the first CO₂-rich stream is produced by passing a carbon dioxide-feed stream through a CO₂-cleaning unit for removing impurities.

5. The method according to claim 1, wherein H₂O is added to the synthesis gas corresponding to a molar percentage of 1.5-3 mol % in the synthesis gas, when the CO₂ content in the synthesis gas is below 0.5 mol %.

6. The method according to claim 1, wherein:
in conducting the CO₂ electrolysis in the step a), the step of separating said first stream comprising CO and CO₂, comprises passing this stream said first stream comprising CO and CO₂ through a CO-enrichment unit, for producing said second stream comprising CO and CO₂, and said second CO₂-rich stream.

7. The method according to claim 1, wherein the step of providing first CO₂-rich stream and passing the first CO₂-rich stream through the first electrolysis unit for producing the first stream comprising CO and CO₂, and the step of providing the water feedstock and passing the water feedstock through the second electrolysis unit for producing the stream comprising H₂, are conducted separately.

8. The method according to claim 1, wherein the step a) comprises by-passing a portion of said first CO₂-rich stream prior to passing said first CO₂-rich stream through said first electrolysis unit.

9. The method according to claim 1, wherein the first electrolysis unit is a solid oxide electrolysis unit, and the second electrolysis unit for producing the stream comprising H₂is: an alkaline/polymer electrolyte membrane electrolysis unit; or a solid oxide electrolysis cell unit.

10. The method according to claim 1, wherein said water feedstock comprises steam produced from other processes of the method.

11. The method according to claim 1, wherein the step of converting the synthesis gas into methanol comprises passing the synthesis gas through a methanol synthesis reactor under the presence of a catalyst for producing a raw methanol stream, optionally further comprises a distillation step of the raw methanol stream for producing a water stream and a separate methanol stream having at least 98 wt % methanol.

12. A method for producing a synthesis gas, comprising:
i) providing a first CO₂-rich stream, providing a water feedstock and combining the water feedstock with the first CO₂-rich stream to form a combined feed gas stream, and passing the combined feed gas stream through an electrolysis unit for producing said synthesis gas which comprises CO, CO₂ and H₂;
wherein the synthesis gas has a module M=(H₂−CO₂)/(CO+CO₂) in the range 1.95-2.10, or a molar ratio H₂/CO of 1.95-2.10,
wherein i) is conducted by once-through co-electrolysis, and
wherein the once-through co-electrolysis is conducted in a solid oxide electrolysis cell unit and the method comprises:
by-passing a portion of said first CO₂-rich stream prior to passing said first CO₂-rich stream through said solid oxide electrolysis cell unit.

13. The method according to claim 12, said synthesis gas has a molar ratio CO/CO₂ of 0.2-0.6, and the method further comprises:
ii) subjecting the synthesis gas to a reverse water gas shift step (rWGS step), optionally in an electrically heated WGS reactor (e-rWGS reactor).

14. The method according to claim 12, further comprising:
iii) converting said synthesis gas into methanol, or synthetic fuels via Fischer Tropsch synthesis (FT synthesis), or methane.

15. The method according to claim 14, comprising converting the synthesis gas into methanol, and wherein H₂O is added to the synthesis gas corresponding to a molar percentage of 1.5-3 mol % in the synthesis gas, when the CO₂ content in the synthesis gas is below 0.5 mol %.

16. A method for producing an alcohol, said alcohol being at least one of C1-C5 alcohols, or for producing methane, comprising the steps of:
a) providing a first CO₂-rich stream and passing the first CO₂-rich stream through a first electrolysis unit for producing a first stream comprising CO and CO₂;
separating from said first stream comprising CO and CO₂:
a second stream comprising CO and CO₂; and
a second CO₂-rich stream; and
recycling the second CO₂-rich stream to said first electrolysis unit;
b) providing a water feedstock and passing the water feedstock through a second electrolysis unit for producing a stream comprising H₂;
c) combining said second stream comprising CO and CO₂ and said stream comprising H₂ into a synthesis gas; and
d) converting said synthesis gas into said alcohol or said methane,
wherein said second stream comprising CO and CO₂ has a molar ratio CO/CO₂ greater than 2.

17. The method according to claim 16, wherein H₂O is added to the synthesis gas corresponding to a molar percentage of 1.5-3 mol % in the synthesis gas, when the CO₂ content in the synthesis gas is below 0.5 mol %.

18. The method according to claim 12, wherein the synthesis gas has a module M=(H₂−CO₂)/(CO+CO₂) in the range 1.95-2.10.

19. The method according to claim 12, wherein the synthesis gas has a molar ratio H₂/CO of 1.95-2.10.
